(19) United States
(12) Patent Application Publication
(10) Pub. No.: US 2012/0196821 A1
(43) Pub. Date: Aug. 2, 2012

(54) METHOD AND FORMULATION FOR THE CONTROL OF PARASITES

(76) Inventors: Roger Mervyn Sargent, Seven Hills (AU); Peter Andrew O'Neill, Seven Hills (AU)

(21) Appl. No.: 13/499,954

(22) PCT Filed: Oct. 18, 2010
(86) PCT No.: PCT/EP2010/065609

§ 371 Date: Apr. 3, 2012

(30) Foreign Application Priority Data
Oct. 19, 2009 (AU) 2009905092
Oct. 19, 2009 (EP) 09173443.4
Oct. 21, 2009 (AU) 2009905174

(51) Int. Cl. A61K 31/7048; A61P 33/00; A61P 33/14

(52) U.S. Cl. 514/30

(57) ABSTRACT

An aqueous micellar formulation for localised topical application to an animal for the concurrent control of internal and external parasites in and on the animal, the formulation comprising a water-miscible co-solvent, a fatty alcohol alkoxylate, a macrocyclic lactone and water. Also described is a method for the concurrent control of internal and external parasites in and on an animal, the method comprising administering to the animal by localised topical application an aqueous micellar formulation comprising a macrocyclic lactone.

### TECHNICAL FIELD

The invention relates to methods and formulations for the control of internal and external parasites in and on an animal.

### BACKGROUND ART

Animals may be affected by a variety of internal and external parasites. Internal and external parasites affecting livestock can have significant detrimental effects on the quality of wool, leather, milk, meat or other products obtained from the livestock and on the general health and well being of the livestock.

A number of formulations containing parasiticides (i.e. anti-parasitic agents) are commercially available. These formulations include formulations for oral administration, for example, as tablets, oral drenches and boli, injectable formulations, and formulations for topical administration. Topical formulations, especially pour-on formulations, are generally easier to administer to an animal than oral formulations or injectable formulations as they require less handling of the animal.

Some of the commercially available topical formulations for the control of ecto-parasites are applied to the majority of the body surface of the animal by jetting or to the entire body surface of the animal by dipping. Other topical formulations for the control of ecto-parasites are applied by pour-on or spot-on application to a portion of the body surface of the animal. These pour-on and spot-on formulations are typically designed to spread the active ingredient over the skin and/or hair of the animal to control the ecto-parasite over the entire body of the animal. Typically the active ingredient is dissolved in the formulation. However, some water-insoluble active ingredients have been formulated as aqueous suspension pour-on formulations for the control of some ecto-parasites, for example, deltamethrin (a synthetic pyrethroid) for the control of lice on sheep (Coopers® Clout®-S, Intervet/Schering-Plough Animal Health) and cattle (Coopers® Easy-Dose, Intervet/Schering-Plough Animal Health) and diflubenzuron (an insect growth regulator) for the control of lice on sheep (Coopers® Magnum®, Intervet/Schering-Plough Animal Health).

Topical formulations for the control of endo-parasites must be able to deliver the active ingredient systemically to the animal to control the parasite, typically by the active ingredient passing through the skin of the animal to the blood stream of the animal. Topical formulations for the control of endo-parasites are generally organic solvent-based formulations. An organic solvent carrier is generally utilised in these formulations to dissolve the active ingredient and to assist the systemic delivery of the active ingredient.

The development of resistance of parasites to the parasiticides used to control them is a continuing problem. For example, there is now widespread resistance of the sheep body louse (Bovicola ovis) to one of the classes of parasiticides used to control it, namely, the synthetic pyrethroids. Accordingly, there is a continuing need to develop alternative methods and formulations for the control of parasites in and on animals.

### SUMMARY OF THE INVENTION

It has now been surprisingly found that internal and external parasites in and on an animal can be concurrently controlled by the localised topical application of an aqueous micellar formulation comprising a macrocyclic lactone. The inventors have found that on localised topical application of an aqueous micellar formulation comprising a macrocyclic lactone, wherein water constitutes at least 50% by weight of the total amount of water and any co-solvents in the formulation, some of the macrocyclic lactone is able to move through the skin of the animal to be absorbed systemically and control internal parasites in the animal and some of the macrocyclic lactone is able to spread over the skin of the animal, or through the fleece or hair of the animal, to control external parasites on the animal. Accordingly, the present invention provides a method for the concurrent control of internal and external parasites in and on animals that can be carried out using a single formulation.

In one aspect, the present invention provides a method for the concurrent control of internal and external parasites in and on an animal, the method comprising administering to the animal by localised topical application an aqueous micellar formulation comprising a macrocyclic lactone.

Typically the formulation comprises a macrocyclic lactone, a surfactant, a water-miscible co-solvent and water, wherein water constitutes at least 50% by weight of the total amount of water and water-miscible co-solvent in the formulation.

In some embodiments, the formulation is administered by applying the formulation in 1, 2 or 3 bands on the back of the animal.

In some embodiments, the formulation comprises two or more macrocyclic lactones.

Typically, the dose of the macrocyclic lactone or mixture of macrocyclic lactones administered to the animal is more than 2.0 mg of the macrocyclic lactones present in the formulation per kg bodyweight of the animal. In some embodiments, the dose administered is more than 4.0 mg of the macrocyclic lactones present in the formulation per kg bodyweight of the animal.

In a second aspect, the present invention provides an aqueous micellar formulation for localised topical application to an animal for the concurrent control of internal and external parasites in and on the animal, the formulation comprising a macrocyclic lactone, a fatty alcohol alkoxylate, a water-miscible co-solvent and water, wherein water constitutes at least 50% by weight of the total amount of water and water-miscible co-solvent in the formulation.

In some embodiments, the formulation comprises from 250 to 450 g/L fatty alcohol alkoxylate.

In a third aspect, the present invention provides an aqueous micellar formulation comprising:

| Ingredient | Amount (g/L) |
|---|---|
| Propylene Glycol | 187.50 ± 10% |
| Fatty alcohol alkoxylate | 375.00 ± 10% |
| Abamectin | 6.00 ± 10% |
| Sodium Dihydrogen Orthophosphate | 7.83 ± 10% |
| Preservative | 1 to 5 |
| Colouring agent | 0 to 5 |
| Water | qs to 1 L |

where the amount of the sodium dihydrogen orthophosphate is calculated based on the weight of the anhydrous salt.

In a fourth aspect, the present invention provides the use of an aqueous micellar formulation comprising a macrocyclic lactone for localised topical application to an animal for the concurrent control of internal and external parasites in and on the animal.

In a fifth aspect, the present invention provides the use of a macrocyclic lactone in the manufacture of an aqueous micellar formulation for localised topical application to an animal for the concurrent control of internal and external parasites in and on the animal.

### DETAILED DESCRIPTION OF THE INVENTION

The present invention is based on the surprising finding that the localised topical application to an animal of an aqueous micellar formulation comprising a macrocyclic lactone is effective in concurrently controlling both external parasites on, and internal parasites in, the animal.

In the method of the present invention, the formulation applied to the animal is an aqueous micellar formulation comprising a macrocyclic lactone. A micellar formulation comprises micelles, i.e. minute colloidal particles. In the aqueous micellar formulation, the macrocyclic lactone, or the majority of the macrocyclic lactone, is present within the core of the micelles in the formulation.

Aqueous micellar formulations comprise a surfactant and water. Typically, the formulation used in the method of the present invention further comprises a water-miscible co-solvent.

The formulation used in the method of the present invention is an “aqueous” formulation. As used herein, by the term “aqueous” in relation to a formulation (e.g. a reference to an “aqueous micellar formulation”), it is meant that the formulation is a liquid formulation comprising water and that water constitutes at least 50% by weight of the total amount of the water and any co-solvent or co-solvents present in the formulation.

A higher concentration of a macrocyclic lactone can be included in an aqueous micellar formulation than can be dissolved in pure water. Aqueous micellar formulations can be prepared that contain an effective amount of a macrocyclic lactone to control internal and external parasites in and on an animal in a volume of the formulation suitable for localised topical application (e.g. for pour-on application). Advantageously, the formulation is an aqueous formulation. Aqueous formulations are generally easier to handle and have less safety and environmental concerns than formulations containing a high proportion of an organic solvent.

The “macrocyclic lactones” are a class of nematicidal and insecticidal compounds derived from Streptomyces spp and active derivatives of such compounds. The macrocyclic lactones include the avermectins and the milbemycins, including active derivatives of the naturally occurring avermectins and milbemycins.

The macrocyclic lactone may be any macrocyclic lactone.

The macrocyclic lactone may be selected from the group of avermectins, including ivermectin (22,23-dihydroavermectin B₁), abamectin, avermectin A₁ₐ, avermectin A_{1b}, avermectin A₂ₐ, avermectin A_{1b}, avermectin B₁ₐ, avermectin B_{1b}, avermectin B₂ₐ and avermectin B_{2b}. The macrocyclic lactone may also be selected from the milbemycins, including moxidectin, milbemycin, milbemycin oxime, milbemycin D (Antibiotic B41 D) and nemadectins. The macrocyclic lactone may also be selected from active derivatives of the naturally occurring avermectins and milbemycins, such as derivatives which have a group at the 25-substituent other than the isopropyl or (S)-sec-butyl groups, as described in European patent application nos. 0214731, 0284176, 0308145, 0317148 and 0335541. The macrocyclic lactone may also be selected from doramectin, selamectin, eprinomectin and emamectin.

Typically, the macrocyclic lactone is an avermectin, more typically, ivermectin or abamectin. Abamectin is a mixture comprising more than 80% avermectin B₁ₐ and less than 20% avermectin B_{1b}.

In some embodiments, the formulation comprises two or more macrocyclic lactones. For example, in some embodiments, the formulation comprises ivermectin and abamectin or comprises abamectin and eprinomectin.

The aqueous micellar formulation comprises a surfactant or mixture of surfactants. Typically the surfactant is a non-ionic surfactant. The non-ionic surfactant may, for example, be selected from sorbitan esters, polyoxyalkylated sorbitan esters, polyoxyalkylated alkyl ethers, polyoxyalkylated fatty alcohols (also known as fatty alcohol alkoxylates), polyoxyalkylated fatty acids, polyalkylene glycol esters, polyoxyalkylated derivatives of castor oil, polyoxyalkylated vegetable oils, polyglycerol esters, copolymers of ethylene oxide and propylene oxide, fatty amine alkoxylates, alkylphenol alkoxylates, alkyl polysaccharides, polymeric surfactants and combinations thereof. The surfactant may also be, or the formulation may also include, an anionic surfactant. Suitable anionic surfactants include linear alkylbenzene sulphonates, C₁₂ to C₁₆ alcohol sulphates, C₁₂ alkoxypolyethanoxy sulphates, alkyl phosphates and phosphonates and combinations thereof.

Preferred surfactants are fatty alcohol alkoxylates (also known as polyalkoxylated fatty alcohols), such as Teric® BL8 and Teric® BL9 (Huntsman Corporation Australia Pty Ltd). Fatty alcohol alkoxylates are a class of surfactant. Fatty alcohol alkoxylates are polyalkylene oxide derivatives of fatty alcohols and comprise a plurality of alkyl oxide groups, typically ethylene oxide and/or propylene oxide groups. Fatty alcohol alkoxylates typically comprise a C₈-C₂₁ branched or linear alkyl group. Typically, fatty alcohol alkoxylates are formed by reacting a fatty alcohol with ethylene oxide and/or propylene oxide at elevated temperatures and pressures in the presence of an acidic or alkaline catalyst.

Preferred fatty alcohol alkoxylates contain at least one propylene oxide group in addition to ethylene oxide groups.

Preferred fatty alcohol alkoxylates can be represented by the formula:

RO-(EO)ₙ(PO)ₘ—H

where R is a branched or straight C₈-C₁₅ alkyl;

EO is —CH₂—CH₂—O—;
each PO is independently selected and is

—CH(CH₃)CH₂—O— or —CH₂CH(CH₃)—O—;
m and n are each at least 1 and m is less than n;
   wherein the individual EO and PO groups may be connected to RO and each other in any order.

In some embodiments, the surfactant is a mixture of fatty alcohol alkoxylates of the above formula.

Preferred surfactants have an HLB (hydrophilic-lipophilic balance) of 10-15.

Fatty alcohol alkoxylates comprising a branched or straight C₈-C₁₅ alkyl are preferred as these surfactants are low foaming and have a low tendency to gel when added to water compared to some other surfactants. These surfactants also have a high cloud point which ensures surfactant/water solubility at temperatures up to 57 to 61° C. These surfactants are also good wetters. Because of the good wetting properties, formulations comprising these surfactants wet wool and hair quickly reducing formulation run-off when the formulation is topically applied to an animal. In addition, the wetting properties of these surfactants facilitate movement of the macrocyclic lactone through the fleece or hair of the animal.

In some embodiments, the amount of surfactant in the formulation is from about 50 to about 450 g/L based on the total formulation. In some embodiments, the amount of surfactant in the formulation is from about 100 to about 450 g/L based on the total formulation. In some embodiments, the amount of surfactant in the formulation is from about 200 to 420 g/L based on the total formulation. In some embodiments, the amount of surfactant in the formulation is from about 300 to 420 g/L based on the total formulation.

The aqueous micellar formulation typically comprises a water-miscible co-solvent, or two or more water-miscible co-solvents.

The co-solvent or co-solvents may be any water-miscible solvent. The co-solvent may, for example, be selected from propylene glycol, glycerol formal, glycerine, benzyl alcohol, glycol ethers, liquid polyethylene glycols, dimethyl sulfoxide, dimethylacetamide, ethyl lactate, dimethyl isosorbide, n-methyl-2-pyrrolidone and mixtures thereof.

In some embodiments, the co-solvent is selected from the group consisting of propylene glycol, glycerol formal, glycerine, benzyl alcohol, glycol ethers, liquid polyethylene glycols, dimethyl sulfoxide, dimethylacetamide, ethyl lactate, dimethyl isosorbide, and mixtures thereof.

Preferred water-miscible co-solvents are propylene glycol and liquid polyethylene glycols, e.g. PEG 200. These co-solvents are preferred as they are non-flammable, non-toxic, minimally irritant and have good regulatory acceptability.

Aqueous micellar formulations comprising a macrocyclic lactone can be unstable, resulting in the degradation of the macrocyclic lactone over time. Without wishing to be bound by theory, the inventors believe that the inclusion of a water-miscible co-solvent, such as propylene glycol, in the aqueous micellar formulation, and maintaining the pH of the formulation at about 5.2 to 7.5, increases the stability of aqueous micellar formulations comprising a macrocyclic lactone. The use of a co-solvent can also assist in the manufacture of the formulation by facilitating the solubilisation of the macrocyclic lactone and preventing or reducing gelling of the surfactant during the preparation of the aqueous micellar formulation. In some embodiments, the amount of co-solvent in the formulation is from 50 to 300 g/L, for example, from 50 to 195 g/L. In some embodiments, the amount of co-solvent in the formulation is from 150 to 200 g/L.

Macrocyclic lactones are active against a variety of endo-parasites. Macrocyclic lactones are, for example, active against Haemonchus, Ostertagia, Trichostrongylus, Nematodirus, Cooperia, Strongyloides, Trichuris, Oesophagostomum, Chabertia and Dictyocaulus species in sheep and against Haemonchus, Ostertagia, Trichostrongylus, Nematodirus, Cooperia, Oesophagostomum and Dictyocaulus species in cattle. Macrocyclic lactones are also active against a variety of external parasites such as lice, ticks and fly larvae on sheep, cattle and other animals and itchmite and nasal bot on sheep. Advantageously, the method and formulation of the present invention can be used to control sheep body lice on sheep, including sheep body lice resistant to deltamethrin and other synthetic pyrethroids.

The concentration of macrocyclic lactone in the formulation is typically selected such that the volume of the formulation required to provide an effective amount of the macrocyclic lactone to control internal and external parasites in and on an animal is a volume convenient for pour-on application, for example, less than 3 mL per kg bodyweight, more typically less than 1.5 mL per kg bodyweight. In some embodiments, the amount of macrocyclic lactone in the formulation is from about 2.5 g/L to about 40 g/L. In some embodiments, the formulation contains an amount of macrocyclic lactone in the range of about 4 g/L to about 30 g/L, about 4 g/L to about 10 g/L or about 4 g/L to about 8 g/L, based on the total formulation.

Typically the formulation comprises:
50-300 g/L water-miscible co-solvent;
50-450 g/L surfactant;
250-890 g/L water;
   wherein water constitutes at least 50% by weight of the total amount of water and water-miscible co-solvent in the formulation.

More typically, the formulation comprises:
150-200 g/L water-miscible co-solvent;
300-420 g/L surfactant;
350-500 g/L water.

In the method of the present invention, the formulation is administered to the animal by localised topical application. “Localised topical application” comprises topically applying the formulation to a minor portion of the body surface of the animal. Typically the formulation is applied to part of the back of the animal, for example, as 1, 2 or 3 lines or bands on the back of the animal.

The formulation is administered to the animal in an effective amount to control internal and external parasites in and on the animal. In some embodiments, the formulation is administered to the animal in an amount to administer to the animal about 0.25 mg to about 50 mg, for example, about 0.4 mg to about 40 mg, of macrocyclic lactone per kg bodyweight. In some embodiments, the formulation is administered to the animal in an amount to administer about 2.0 mg to about 10.0 mg, for example, about 2.5 mg to about 8.0 mg, of macrocyclic lactone per kg bodyweight. However, as will be apparent to a person skilled in the art, the effective amount to control particular internal and external parasites in and on an animal will vary depending on a number of factors including, for example, the particular macrocyclic lactone or macrocyclic lactones in the formulation, the species of animal, the age, sex and health of the animal, the particular parasites infesting the animal and the severity of parasitic infestation. For any given case, a person skilled in the art could readily determine an effective amount to control parasites in and on an animal.

The animal may be any animal. The animal is typically a mammal. The animal may, for example, be a domestic animal such as a sheep, cow, goat, horse, donkey, mule, llama, alpaca or pig, or a zoo animal. The animal may also, for example, be a companion animal such as a cat or dog. When the animal is a sheep, the aqueous micellar formulation is preferably administered to the sheep within a week, more preferably, within 24 hours, of shearing.

The inventors have found that the localised topical application of an aqueous micellar formulation comprising a macrocyclic lactone, wherein water constitutes at least 50% by weight of the total amount of water and any co-solvents in the formulation, is able to control both internal and external parasites in and on an animal. The inventors have found that on localised topical application of the aqueous micellar formulation, some of the macrocyclic lactone is able to move through the skin of the animal to be absorbed systemically and control internal parasites in the animal. In addition, the macrocyclic lactone is also able to spread over the skin of the animal, or through the fleece or hair of the animal, to control external parasites on the animal. Without wishing to be bound by theory, the inventors believe that the combination of the presence of the surfactant in the formulation and the water forming the majority of the water and co-solvents present in the formulation, facilitates the spreading of the macrocyclic lactone over the skin or through the fleece or hair of the animal. Formulations comprising a fatty alcohol alkoxylate surfactant, where the fatty alcohol alkoxylate comprises a C₈-C₁₅ alkyl, are preferred as these surfactants have good wetting properties reducing run-off of the formulation when applied to the animal and facilitating the penetration of the macrocyclic lactone through the skin as well as facilitating the spreading of the macrocyclic lactone over the skin, or through the fleece or hair, of the animal.

The inventors have also found that aqueous micellar formulations comprising a macrocyclic lactone are also advantageously “rainfast”, that is, the formulation is effective in controlling internal and external parasites in and on an animal even when applied to a wet animal or when the animal is exposed to rain shortly after topical application of the formulation. Without wishing to be bound by theory, the inventors believe the aqueous nature of the formulation contributes to the rainfast properties of the formulation. As the formulation is an aqueous formulation, the formulation is able to mix with water present on the skin, fleece or hair of the animal. As a result, when the formulation is topically applied to a wet animal, or if the animal is exposed to rain soon after topical application of the formulation, the formulation is still able to spread over the skin of the animal, or through the fleece or hair of the animal, and control internal and external parasites in and on the animal. The rainfast properties of the formulation are a significant advantage for use in the grazing industry. A significant disadvantage with some organic solvent-based formulations is that they are not rainfast, which limits their use in large scale grazing operations.

The inventors have found that a further advantage of controlling internal and external parasites in and on an animal using the method of the present invention is the low residues of the macrocyclic lactone in the wool or hair on the animal within a few months after treatment due to the degradation of the macrocyclic lactone when exposed to sunlight. A disadvantage of some prior art topical formulations for the control of external parasites on sheep and other wool-producing animals is that significant amounts of the active ingredient remain in the wool for up to a year, which can cause adverse environmental issues when the active ingredient is removed from the wool during scouring of the wool.

Typically, the aqueous micellar formulation comprises one or more of a preservative and a buffer to extend the shelf life of the formulation.

Suitable buffers will maintain the pH of the formulation in the range of about 5.2 to about 7.5, more preferably about 5.7 to about 7.0. Suitable buffers include soluble monobasic and/or dibasic phosphates, such as sodium dihydrogen orthophosphate. The buffer may, for example, be present in the formulation in an amount of about 0.2 to about 20 g/L, for example, about 5 to about 10 g/L, based on the total formulation.

The preservative may, for example, be selected from diazolidinyl urea, sodium methyl hydroxybenzoate, sodium propyl hydroxybenzoate, 1,2-benzisothiazolin-3-one and sodium hydroxymethylglycinate. The preservative may, for example, be present in the formulation in an amount of about 1 g/L to about 5 g/L based on the total formulation.

The aqueous micellar formulation typically comprises a colouring agent. Colouring agents enable treated mammals to be readily distinguished from untreated animals. The colouring agent may be dissolved, suspended or dispersed in the formulation. The nature of the colouring agent is unimportant and a wide variety of suitable dyes and pigments will be known to a person skilled in the art. The colouring agent may be soluble or insoluble in water. Generally, however, the colouring agent will be biodegradable so as to fade and not permanently mark the skin or fleece or scourable so that it can be removed from wool during processing. Some examples of suitable colouring agents include: FD&C Brilliant Blue No. 1 (Brilliant Blue FCF, Hexacol Brilliant Blue), Fast Scarlet Pigment 3610 (Dispers Scarlet FK3610), Luconyl Green FK872, Fluoresceine LT, Tartrazine, Carmine Dispersion R1276 and C.I. Food Red (Carmosine, CI 14720).

The formulation may also comprise other veterinary acceptable excipients such as viscosity modifiers. The formulation may also comprise active ingredients in addition to active ingredients selected from the macrocyclic lactones. In some embodiments, the formulation does not comprise any parasiticides other than one or more parasiticides selected from the macrocyclic lactones. In other embodiments, the formulation comprises, in addition to one or more parasiticides selected from the macrocyclic lactones, one or more parasiticides that are not a macrocyclic lactone.

The aqueous micellar formulation comprising a macrocyclic lactone may be prepared by methods and techniques known in the art for preparing an aqueous micellar formulation.

An aqueous micellar formulation comprising a macrocyclic lactone, a surfactant and a water-miscible co-solvent may, for example, be prepared by the following process:

### Step 1

Mix the co-solvent and surfactant.

### Step 2

Add the macrocyclic lactone or macrocyclic lactones to the mixture of the co-solvent and surfactant and mix (e.g. by stirring) until the macrocyclic lactone(s) have been dissolved.

### Step 3

Add the water, optionally with a buffer, preservative and colouring agent, and mix (e.g. by stirring) to form an aqueous micellar formulation.

The method of the present invention may be used to control internal and external parasites sensitive to the macrocyclic lactone or macrocyclic lactones in the formulation. The class of macrocyclic lactones is particularly effective against nematodes (internal parasites) and lice, ticks and mites (external parasites). A preferred macrocyclic lactone for use in the present invention is abamectin. An aqueous micellar formulation of abamectin may, for example, be used in the method of the present invention for the concurrent control of body lice (Bovicola ovis), including lice resistant to synthetic pyrethroids or tolerant to insect growth regulators, and abamectin-sensitive gastrointestinal nematodes, including benzimidazole, levamisole and morantel resistant strains, on and in sheep. Such a formulation may be used to control, amongst other parasites, the following abamectin-sensitive gastrointestinal nematodes and ectoparasites in and on sheep and, at a dose of about 3 mg/kg abamectin, is effective for up to 28 days after treatment:
Barber's Pole Worm (Haemonchus contortus) including inhibited L₄ stage,
Large Stomach Worm (Haemonchus placei),
Small Brown Stomach Worm (Teladorsagia circumcincta), (including inhibited L₄ stage),
Stomach Hair Worm (Trichostrongylus axei),
Black Scour Worm (Trichostrongylus spp),
Small Intestinal Worm (Cooperia spp),
Thin-necked Intestinal Worm (Nematodirus spp),
Large Mouthed Bowel Worm (Chabertia ovine),
Nodule Worm (Oesophagostomum columbianum),
Large Bowel Worm (Oesophagostomum venulosum),
Whipworm (Trichuris ovis),
Intestinal Threadworm (Strongyloides papillosus),
Nasal Bot (Oestrus ovis) (parasitic larval stages)
Large Lungworm (Dictyocaulus filaria) and
Itchmite (Psorergates ovis)

### EXAMPLES

Embodiments of the invention are described below, by way of example only, with reference to the following examples.

### Example 1

A formulation comprising the following ingredients was prepared as described below.

| | | | Amount |
|---|---|---|---|
| Item | Ingredient | Function | (g/L) |
| 1 | Propylene Glycol USP | Co-solvent | 187.50 |
| 2 | Teric BL8 (a fatty alcohol | Surfactant | 375.00 |
| | alkoxylate) | | |
| 3 | Abamectin | Active | 6.00 |
| 4 | Sodium Dihydrogen | Buffer | 7.83 |
| | Orthophosphate—anhydrous | | |
| 5 | Diazolidinyl Urea (Germall II) | Preservative | 2.00 |
| 6 | Brilliant Blue FCF (CI. 142090) | Dye | 0.182 |
| 7 | C.I. Food Red | Dye | 0.078 |
| | (Carmosine, CI. 14720) | | |
| 8 | Purified Water | Diluent | qs to 1 L |

### Method of Manufacture

1. Add the PROPYLENE GLYCOL (Item 1) and TERIC BL8 (Item 2) to the manufacturing vessel equipped with a stirrer, turn the stirrer on and mix.
2. Add with stirring the ABAMECTIN (Item 3) and stir until all the ABAMECTIN has dissolved.
3. To a separate vessel equipped with a stirrer transfer a portion of the PURIFIED WATER (Item 8), SODIUM DIHYDROGEN ORTHOPHOSPHATE (Item 4) and GERMALL II (Item 5) and stir until dissolved.
4. Transfer the solution from Step 3 to the batch and stir. Rinse the vessel with a portion of the PURIFIED WATER (Item 8) and transfer to the batch and continue stirring.
5. In a suitable container pre-dissolve the BRILLIANT BLUE FCF (Item 6) and C.I. FOOD RED (CARMOSINE) (Item 7) in a portion of the PURIFIED WATER (Item 8) and add to the vessel and stir.
6. Submit a sample to the lab for a pH check. Required pH range=5.7 to 7.0 (Adjustment, if required, to be carried out with either SODIUM HYDROXIDE or SODIUM DIHYDROGEN ORTHOPHOSPHATE solutions).
7. Dilute batch to volume with remaining PURIFIED WATER (Item 8) and stir for 15 minutes.
8. Filter through a suitable 10-50 μm filter to a stainless steel holding tank in readiness for filling.

### Stability

Suitable specifications for the formulation at release and expiry are as follows:

| APPEARANCE: | A clear dark blue coloured | |
|---|---|---|
| | liquid (Release and Expiry) | |
| ABAMECTIN | 5.70-6.30 g/L | (Release) |
| CONTENT: | 5.40-6.60 g/L | (Expiry) |
| SG (20° C.): | 1.020-1.050 | (Release and Expiry) |
| PH: | 5.7-7.0 | (Release) |
| | 5.2-7.5 | (Expiry) |

The accelerated and real time stability of the formulation was tested by storing samples of the formulation in High Density Polyethylene (HDPE) bottles and backpacks at 4° C., 30° C./65% Relative Humidity (RH) and 40° C./75% RH. At various times after storage the samples were removed and analysed for abamectin content, appearance and pH.

After 1461 days storage at 4° C., 30° C./65% RH and 40° C./75% RH, all samples of the formulation tested remained within the expiry specifications for abamectin content, appearance and pH.

The study was continued. After 1832 days storage at 4° C. and 30° C./65% RH, all samples of the formulation tested remained within the expiry specifications for abamectin content, appearance and pH.

Based on the real time stability data generated for the formulation, a five year shelf life when stored in a closed container below 30° C. has been demonstrated.

### Example 2

### Efficacy, Safety and Wool Residue Studies (3 mg/kg Abamectin)

### Nematode Efficacy Studies

The following studies were conducted concerning the nematode efficacy of a single volume application of 5 mL/10 kg (3 mg/kg abamectin) of the formulation of Example 1 to sheep:

### 1. Pen Trial—Artificial Infestation—Faecal Egg Count Reductions

Forty, 12 month old, lousy, merino hoggets were artificially infested with the viable infective larvae (L₃) of Oesophogostomum columbianum (1,000), Haemonchus contortus (4,000) and Trichostrongylus colubriformis (8,000). On day −1 of the trial the animals were divided into four groups of ten animals such that each group had a similar mean lice and nematode burden. Within the 24 hours following shearing the animals in Group 2 were weighed and treated by pour-on application of the formulation of Example 1 to the back of the animal at the rate of 3 mg/kg (5 mL/10 kg) using a 7-hole T-bar applicator. Group 3 and 4 were weighed and treated by pour-on application of a similar formulation containing 9.0 g/L abamectin to the back of the animal at the rate of 4.5 mg/kg (5 mL/10 kg) using a 7-hole T-bar applicator. Group 1 was left untreated.

Faecal samples were collected from Groups 1-3 on days 7, 14, 21 and 28 post treatment and lice counts were conducted on these groups on days 7, 21, 42, 84 and 149 post treatment.

### 2. Critical Slaughter Trial 1—Artificial Infestation—Total Worm Counts

Thirty three, 18-24 month old merino wethers were artificially infested with the viable infective larvae (L₃) of Oesophogostomum columbianum (1,000), Haemonchus contortus (8,000) and Trichostrongylus colubriformis (16,000). On day −3 of the trial the animals were divided into three groups of 10 animals. Within the 24 hours following shearing the animals in Group 1 were weighed and treated from the poll to the tail with the formulation of Example 1 at the rate of 5 mL/10 kg via a pour-on applicator with a 7-hole T-bar nozzle. Group 2 was treated orally with a commercially-available oral drench (WSD ABAMECTIN ORAL DRENCH FOR SHEEP AND LAMBS) at the registered dose rate (0.2 mg/kg) and Group 3 was left untreated.

11 days post-treatment each of the animals had faecal samples taken before being slaughtered on days 12-13 for collection of their abomasum, small intestine and large intestine for total worm counts.

### 3. Critical Slaughter Trial 2—Artificial Infestation—Total Worm Counts

Thirty two, 7 month old cross-bred wethers were artificially infested with the viable infective larvae (L₃) of Oesophogostomum columbianum (1,000), Haemonchus contortus (8,000) and Trichostrongylus colubriformis (16,000). On day −3 of the trial the animals were divided into three groups of 10 animals. Within the 24 hours following shearing the animals in Group 1 were weighed and treated from the poll to the tail with the formulation of Example 1 at the rate of 5 mL/10 kg via a pour-on applicator with a 7-hole T-bar nozzle. Group 2 was treated orally with WSD ABAMECTIN ORAL DRENCH FOR SHEEP AND LAMBS at the registered dose rate (0.2 mg/kg) and Group 3 was left untreated.

11 days post-treatment each of the animals had faecal samples taken before being slaughtered on days 12-13 for collection of their abomasum, small intestine and large intestine for total worm counts.

### 4. Rainfastness Trial—Artificial Infestation—Faecal Egg Count Reductions

Thirty, lice infested merino sheep were artificially infested with the viable infective larvae (L₃) of Oesophogostomum columbianum (1,000), Haemonchus contortus (4,000) and Trichostrongylus colubriformis (8,000). On day −2 of the trial the animals were divided into five groups of 6 animals with similar mean lice and nematode burdens. Within the 24 hours following shearing, Groups 3 to 5 were weighed and treated from the poll to the tail with the formulation of Example 1 at the rate of 5 mL/10 kg via a pour-on applicator with a 7-hole T-bar nozzle. Groups 1 and 2 remained untreated as negative controls. On the day of treatment, Groups 2, 4 and 5 were also exposed to 25 mm of artificial rain, Groups 2 and 4 one hour prior to treatment and Group 5 one hour after treatment.

In addition to the pre-treatment lice and faecal egg counting, the sheep had lice counts done at 20, 42, 88 and 145 days post-treatment and faecal egg counts done at 8, 15, and 28 days post-treatment.

### 5. Field Trials—Natural Infestation—Faecal Egg Count Reductions

Three field efficacy trials were conducted to assess the nematode efficacy of the formulation of Example 1 under field conditions. The trials were conducted in Southern Queensland (2 sites) and Southern New South Wales (1 site) on properties with a concurrent lice infestation. A total of 2130 nematode infested sheep were treated over the three sites.

As the properties had a concurrent lice infestation and lice efficacy data for the formulation of Example 1 was also being generated at the sites it was not possible to have untreated control sheep included in the trials.

At each field trial site, within the 24 hours following shearing, the trial sheep were treated from the poll to the tail with the formulation of Example 1 via a pour-on applicator with a 7-hole T-bar nozzle according to the dose break table shown below (Table 1).

TABLE 1
Dose Break Table

| Body Weight (kg) | Dose (mL) |
|---|---|
| 10 to 15 | 7.5 |
| 16 to 20 | 10 |
| 21 to 30 | 15 |
| 31 to 40 | 20 |
| 41 to 50 | 25 |
| 51 to 60 | 30 |
| 61 to 70 | 35 |

Above 70 kg treat sheep at 5 mL per 10 kg

Faecal samples for individual faecal egg counts and bulking for larval differentiation were collected from the tracer animals on days 0, 7, 14, 21, 28, 42 and 56 approximately. For two sites, 25 tracer sheep were used, and for the third site, 50 tracer sheep were used.

### Results of Nematode Efficacy Studies

Nematode efficacy was confirmed to be approximately 100% for all nematodes evaluated in the two critical slaughter trials (Table 2).

TABLE 2
Summary of Results of Critical Slaughter Trials (for nematodes)

| | | | | |
|---|---|---|---|---|
| | | Mean % | Mean % | Mean % |
| | Mean % | Reduction | Reduction | Reduction |
| | Reduction | Haemon- | Trichos- | Oesophogo- |
| Trial | Egg Count | chus | trongylus | stomum |

Arithmetic

| | | | | |
|---|---|---|---|---|
| Critical Slaughter | 100.0 | 100.0 | >99.9 | 100.0 |
| Trial 1 | | | | |
| Critical Slaughter | 100.0 | 100.0 | 100.0 | 100.0 |
| Trial 2 | | | | |

Geometric

| | | | | |
|---|---|---|---|---|
| Critical Slaughter | 100.0 | 100.0 | >99.9 | 100.0 |
| Trial 1 | | | | |
| Critical Slaughter | 100.0 | 100.0 | 100.0 | 100.0 |
| Trial 2 | | | | |

Nematode efficacy was confirmed to be high in the Pen Trial and Rainfastness Trial (both involving penned animals) with the efficacy being reduced only when sheep were wet after treatment (Table 3).

TABLE 3
Summary of Results of Pen Trial and
Rainfastness Trial (for nematodes)

| | Mean % Reduction in Egg Count | | | |
|---|---|---|---|---|
| Trial | ~Day 7 | ~Day 14 | ~Day 21 | ~Day 28 |

Arithmetic

| | | | | |
|---|---|---|---|---|
| Pen Trial | >99.9 | >99.9 | >99.9 | >99.9 |
| Rainfastness | 97.7 | 90.5 | 94.6 | 65.4 |
| Trial—Dry Group | | | | |
| Rainfastness | 95.9 | 89.3 | 86.6 | 85.9 |
| Trial—Wet Before | | | | |
| Rainfastness | 84.9 | 50.6 | 61.2 | 27.4 |
| Trial—Wet After | | | | |

Geometric

| | | | | |
|---|---|---|---|---|
| Pen Trial | >99.9 | >99.9 | >99.9 | >99.9 |
| Rainfastness | 99.8 | 97.7 | 96.3 | 83.2 |
| Trial—Dry Group | | | | |
| Rainfastness | 99.7 | 95.6 | 94.9 | 90.1 |
| Trial—Wet Before | | | | |
| Rainfastness | 93.0 | 56.8 | 66.9 | 63.5 |
| Trial—Wet After | | | | |

In the field trials the efficacy ranged from 95-100% for up to 28 days, longer in some trials. The treatment efficacies based on faecal egg counts for the formulation in the field trials are presented in Table 4.

TABLE 4
Summary of Results of Field Trials (for nematodes)

| | ~Day | ~Day | ~Day | ~Day | ~Day | ~Day |
|---|---|---|---|---|---|---|
| Trial Site | 7 | 14 | 21 | 28 | 42 | 56 |

% Nematode Efficacy—Arithmetic Means

| | | | | | | |
|---|---|---|---|---|---|---|
| St George, QLD | 99.0 | 98.7 | 98.1 | 95.9 | 82.3 | — |
| (697 Merino | | | | | | |
| ewes) | | | | | | |
| St George, QLD | 100.0 | 100.0 | 100.0 | 99.8 | 99.7 | 99.6 |
| (1060 Merino | | | | | | |
| wethers) | | | | | | |
| Ganmain, NSW | 97.9 | 96.7 | 97.7 | 98.7 | 99.7 | 96.1 |
| (373 Merino | | | | | | |
| ewes) | | | | | | |

% Nematode Efficacy—Geometric Means

| | | | | | | |
|---|---|---|---|---|---|---|
| St George, QLD | 99.8 | 99.6 | 99.6 | 96.8 | 84.8 | — |
| (697 Merino | | | | | | |
| ewes) | | | | | | |
| St George, QLD | 100.0 | 100.0 | 100.0 | >99.9 | >99.9 | >99.9 |
| (1060 Merino | | | | | | |
| wethers) | | | | | | |
| Ganmain, NSW | 99.6 | 99.6 | 99.7 | 99.7 | 99.9 | 99.2 |
| (373 Merino | | | | | | |
| ewes) | | | | | | |

No treatment related adverse events were observed during any of the trials.

### Lice Efficacy Studies

The following studies were conducted concerning the lice efficacy of a single volume application of 5 mL/10 kg (3 mg/kg abamectin) of the formulation of Example 1 to sheep:

### 1. Pen Trial—Natural Infestation

In the Pen Trial described above (Study 1), lice counts were conducted on the sheep of Groups 1, 2 and 3 at 7, 21, 42, 84 and 149 days post treatment.

### 2. Rainfastness Trial—Natural Infestation

In the Rainfastness Trial described above (Study 4), lice counts were conducted on the sheep of Groups 1, 2, 3, 4 and 5 at 20, 42, 88 and 145 days post-treatment.

### 3. Field Trials—Natural Infestation

Seven field efficacy trials were conducted to assess the lice efficacy of the formulation of Example 1 under field conditions. The trials were conducted in Southern Queensland (2 sites) and Southern New South Wales (2 sites), Southern Western Australia (2 sites) and Northern Victoria (1 site) on commercial properties with moderate to heavy lice infestations. A total of 5301 lice infested sheep were treated over the seven sites.

At each field trial site, within the 24 hours following shearing, the trial sheep were treated from the poll to the tail with the formulation of Example 1 via a pour-on applicator with a 7-hole T-bar nozzle according to the dose break table shown below (Table 5).

TABLE 5
Dose Break Table

| Body Weight (kg) | Dose (mL) |
|---|---|
| 10 to 15 | 7.5 |
| 16 to 20 | 10 |
| 21 to 30 | 15 |
| 31 to 40 | 20 |
| 41 to 50 | 25 |
| 51 to 60 | 30 |
| 61 to 70 | 35 |

Above 70 kg treat sheep at 5 mL per 10 kg

In addition to the pre-treatment lice counts, lice counts were conducted on 25 tracer animals on days 7, 21, 42, 84 and 150 approximately. At the 150 day lice counts an additional 25 sheep were counted, these sheep were selected from the rest of the flock based on signs of visible fleece derangement or rubbing etc. At sites where no treatments with lousicidal activity were applied to the trial sheep between 150 days and the next shearing another lice count was conducted prior to shearing.

### Results of Lice Efficacy Studies

Lice efficacy was confirmed to be 100% at each count in the Pen Trial and Rainfastness Trial with the efficacy being unaffected by wetting prior to or after treatment (Table 6).

TABLE 6
Summary of Results of Pen Trial and Rainfastness Trial (for lice)

| | Mean % Reduction in Lice Count | | | | |
|---|---|---|---|---|---|
| | ~Day | ~Day | ~Day | ~Day | ~Day |
| Trial | 7 | 21 | 42 | 84 | 150 |

Arithmetic

| | | | | | |
|---|---|---|---|---|---|
| Pen Trial | 100.00 | 100.00 | 100.00 | 100.00 | 100.00 |
| Rainfastness | — | 100.00 | 100.00 | 100.00 | 100.00 |
| Trial—Dry Group | | | | | |
| Rainfastness | — | 100.00 | 100.00 | 100.00 | 100.00 |
| Trial—Wet Before | | | | | |
| Rainfastness | — | 100.00 | 100.00 | 100.00 | 100.00 |
| Trial—Wet After | | | | | |

Geometric

| | | | | | |
|---|---|---|---|---|---|
| Pen Trial | 100.00 | 100.00 | 100.00 | 100.00 | 100.00 |
| Rainfastness | — | 100.00 | 100.00 | 100.00 | 100.00 |
| Trial—Dry Group | | | | | |
| Rainfastness | — | 100.00 | 100.00 | 100.00 | 100.00 |
| Trial—Wet Before | | | | | |
| Rainfastness | — | 100.00 | 100.00 | 100.00 | 100.00 |
| Trial—Wet After | | | | | |

Over 5000 sheep were treated in the seven lice field efficacy studies. These studies were conducted on sheep within 24 hours of shearing. Up to approximately 150 days after treatment, efficacy on the 25 tracer animals ranged from 99.2 to 100.0% (arithmetic and geometric means) excluding one site where early lambing compromised the trial however, even at this site the efficacy was 97.3 and 98.9% based on arithmetic and geometric means respectively. Up to approximately 150 days after treatment, efficacy (relative to the pre-treatment counts of the 25 tracers) on an additional 25 sheep selected from the flock on the basis of visible fleece derangement ranged from 96.6 to 100.0% (arithmetic and geometric means) excluding the arithmetic means at two sites of 86.4 (early lambs) and 92.9%. At two sites the lice problem was due to treatment failures with currently registered IGR treatments; the results at these sites confirmed that the formulation is effective against IGR tolerant lice.

The treatment efficacies against lice for the formulation in the field trials are presented in Table 7 below.

TABLE 7
Summary of Results of Field Trials (for lice)

| | ~Day | ~Day | ~Day | ~Day | ~Day | ~Day 150 | Shear- |
|---|---|---|---|---|---|---|---|
| Trial Site | 7 | 21 | 42 | 84 | 150 | Extra 25 | ing |

% Lice Efficacy—Arithmetic Means

| | | | | | | | |
|---|---|---|---|---|---|---|---|
| St George, | 99.6 | 100.0 | 100.0 | 100.0 | 99.9 | 99.9 | 99.5 |
| QLD (697 | | | | | | | |
| Merino ewes) | | | | | | | |
| Kojonup, WA | 99.6 | 100.0 | 100.0 | 100.0 | 99.9 | 92.9 | — |
| (937 Merino | | | | | | | |
| ewes and | | | | | | | |
| wethers)** | | | | | | | |
| Howlong, | 99.7 | >99.9 | 100.0 | 99.9 | 99.8 | 99.8 | — |
| NSW (678 | | | | | | | |
| Merino ewes | | | | | | | |
| and 15 | | | | | | | |
| Merino rams) | | | | | | | |
| Kojonup, WA | 93.6 | 99.9 | 100.0 | 100.0 | 99.2 | 96.8 | — |
| (736 Merino | | | | | | | |
| ewes)** | | | | | | | |
| Violet Town, | — | — | 99.2 | 99.9 | 97.3 | 86.4 | — |
| VIC (789 | | | | | | | |
| Merino ewes | | | | | | | |
| and 16 | | | | | | | |
| Merino rams) | | | | | | | |
| St George, | 100.0 | 100.0 | 100.0 | 100.0 | 100.0 | 100.0 | 100.0* |
| QLD (1060 | | | | | | | |
| Merino | | | | | | | |
| wethers) | | | | | | | |
| Ganmain, | 99.5 | 100.0 | 100.0 | 100.0 | 100.0 | 100.0 | — |
| NSW (373 | | | | | | | |
| Merino ewes) | | | | | | | |

% Lice Efficacy—Geometric Means

| | | | | | | | |
|---|---|---|---|---|---|---|---|
| St George, | 99.7 | 100.0 | 100.0 | 100.0 | 99.9 | 99.9 | 99.5 |
| QLD (697 | | | | | | | |
| Merino ewes) | | | | | | | |
| Kojonup, WA | 99.8 | 100.0 | 100.0 | 100.0 | 99.9 | 98.5 | — |
| (937 Merino | | | | | | | |
| ewes and | | | | | | | |
| wethers)** | | | | | | | |
| Howlong, | 99.8 | >99.9 | 100.0 | 99.9 | 99.9 | 99.8 | — |
| NSW (678 | | | | | | | |
| Merino ewes | | | | | | | |
| and 15 | | | | | | | |
| Merino rams) | | | | | | | |
| Kojonup, WA | 96.1 | 99.9 | 100.0 | 100.0 | 99.6 | 97.7 | — |
| (736 Merino | | | | | | | |
| ewes)** | | | | | | | |
| Violet Town, | — | — | 99.6 | 99.9 | 98.9 | 96.6 | — |
| VIC (789 | | | | | | | |
| Merino ewes | | | | | | | |
| and 16 | | | | | | | |
| Merino rams) | | | | | | | |
| St George, | 100.0 | 100.0 | 100.0 | 100.0 | 100.0 | 100.0 | 100.0* |
| QLD (1060 | | | | | | | |
| Merino | | | | | | | |
| wethers) | | | | | | | |
| Ganmain, | 99.4 | 100.0 | 100.0 | 100.0 | 100.0 | 100.0 | — |
| NSW (373 | | | | | | | |
| Merino ewes) | | | | | | | |

Notes:
The post-treatment lice counts at site 5 were conducted on days 75, 126 and 159.
*= 281 day count not pre-shearing count.
**= Combined efficacy of two flocks.

No treatment related adverse events were observed during any of the nematode efficacy or lice efficacy studies. No adverse effects on the skin or fleece were noted in any of the studies apart for a slight affect on three tracer sheep in a field trial which may or may not have been treatment related and resolved with 43 days of treatment.

### Wool Residues Study

A wool residue band sampling trial was conducted with the formulation of Example 1 which showed that the residues of abamectin in wool following treatment at 5 mL/10 kg (3 mg/kg) were less than 0.5 mg/kg 48 days after treatment and below the Limit of Detection (LOD) of the analytical method (0.005 mg/kg) for four of the five sheep 134 days post-treatment and all five sheep 181 days post-treatment. Analysis of core samples collected from field trial sites confirmed the band sampling results with abamectin residues in the core samples being close to the LOD of 0.005 mg/kg at most sites.

### Safety Study

A Margin of Safety Study was conducted with 1×, 3× and 5× the dose rate of the formulation of Example 1 used in the above studies (i.e. 1×, 3× and 5×5 mL/10 kg). Sheep were observed at 14, 7 and 1 days prior to treatment, as well as at 1, 4 and 7 hours post-treatment and then at 1, 2, 7, 9 and 14 days post-treatment. Observations were made of the gastrointestinal, cardiovascular, and respiratory systems along with feeding behaviour, body weight, temperature, general behaviour, neuromuscular function, morbidity, mortality and skin/hide irritancy. Haematological and biochemical analysis of blood samples was also conducted. Statistical analysis showed that the data collected before treatment was not significantly different to the data collected post-treatment for the majority of traits observed or measured. Similarly, there were few differences between the groups receiving different treatment rates.

A similar safety study in which the formulation of Example 1 was administered to sheep at a higher dose rate of 50 mL/10 kg (30 mg abamectin per kg bodyweight) demonstrated the formulation was also safe at this dose rate.

### Example 3

### Efficacy, Safety and Wool Residue Studies (6 mg/kg Abamectin)

The following studies were carried out using the formulation of Example 1 administered at twice the dose rate of the nematode efficacy studies and lice efficacy studies of Example 2, that is, administered at a dose rate of 10 mL/10 kg (6 mg/kg abamectin).

### Nematode Efficacy Studies

Four field trial studies were conducted concerning the nematode efficacy of the double volume application of the formulation on naturally infested sheep. These field trials followed the same general design as the field trials described in Example 2. The results are summarised below in Table 8. The nematode compositions at the four trial sites is summarised in Table 9 below.

TABLE 8
Summary of Results of Field Trials (for nematodes)

| | ~Day | ~Day | ~Day | ~Day |
|---|---|---|---|---|
| Trial Site | 7 | 14 | 21 | 28 |

% Nematode Efficacy—Arithmetic Means

| | | | | |
|---|---|---|---|---|
| Crookwell, NSW (421 mixed | 99.25 | 98.44 | 98.88 | 90.26 |
| sex Merino hoggets) | | | | |
| Chakola, NSW (327 Merino | 99.36 | 98.72 | 88.44 | 88.65 |
| wethers) | | | | |
| Lochaber, SA (400 Merino | — | 99.92 | 99.08 | 93.61 |
| wethers) | | | | |
| Forbes, NSW (358 Merino | 98.99 | 98.32 | 100.00 | 100.00 |
| ewes) | | | | |

% Nematode Efficacy—Geometric Means

| | | | | |
|---|---|---|---|---|
| Crookwell, NSW (421 mixed | 99.95 | 99.77 | 99.85 | 97.84 |
| sex Merino hoggets) | | | | |
| Chakola, NSW (327 Merino | 99.89 | 99.55 | 98.84 | 99.32 |
| wethers) | | | | |
| Lochaber, SA (400 Merino | — | 99.97 | 99.65 | 94.81 |
| wethers) | | | | |
| Forbes, NSW (358 Merino | 99.56 | 99.33 | 100.00 | 100.00 |
| ewes) | | | | |

TABLE 9
Nematode Composition

| | Nematode Composition (%) | | | |
|---|---|---|---|---|
| | | Tricho- | | Oesophogo- |
| Trial Site | Haemonchus | strongylus | Ostertagia | stomum |
| Crookwell, NSW | — | 78 | 22 | — |
| (421 mixed sex | | | | |
| Merino hoggets) | | | | |
| Chakola, NSW | 78 | 20 | 2 | — |
| (327 Merino | | | | |
| wethers) | | | | |
| Lochaber, SA (400 | 1 | 49 | 10 | 40 |
| Merino wethers) | | | | |
| Forbes, NSW (358 | 4 | 68 | 23 | 5 |
| Merino ewes) | | | | |

### Lice Efficacy Studies

Five field studies were conducted concerning the lice efficacy of the double volume application of the formulation. These field trials followed the same general design as the field trials described in Example 2. The results are summarised below in Table 10.

TABLE 10
Summary of Results of Field Trials (for lice)

| | | | | | | ~Day |
|---|---|---|---|---|---|---|
| | ~Day | ~Day | ~Day | ~Day | ~Day | 150 Ex- |
| Trial Site | 7 | 21 | 42 | 84 | 150 | tra 25 |

% Lice Efficacy—Arithmetic Means

| | | | | | | |
|---|---|---|---|---|---|---|
| Crookwell, | 99.65 | 100.00 | 100.00 | 99.95 | 100.00 | No Lice |
| NSW (421 | | | | | | |
| mixed sex | | | | | | |
| Merino | | | | | | |
| hoggets) | | | | | | |
| Chakola, NSW | 97.13 | 100.00 | 100.00 | 100.00 | 100.00 | No Lice |
| (327 Merino | | | | | | |
| wethers) | | | | | | |
| York, WA (305 | 99.96 | 100.00 | 100.00 | 100.00 | 100.00 | No Lice |
| Merino ewes) | | | | | | |
| Lochaber, SA | 99.96 | 99.96 | 100.00 | 100.00 | 100.00 | No Lice |
| (400 Merino | | | | | | |
| wethers) | | | | | | |
| Forbes, NSW | 99.34 | 100.00 | 100.00 | 100.00 | 100.00 | No Lice |
| (358 Merino | | | | | | on 24 of |
| ewes) | | | | | | 25 sheep* |

% Lice Efficacy—Geometric Means

| | | | | | | |
|---|---|---|---|---|---|---|
| Crookwell, | 99.74 | 100.00 | 100.00 | 99.96 | 100.00 | No Lice |
| NSW (421 | | | | | | |
| mixed sex | | | | | | |
| Merino | | | | | | |
| hoggets) | | | | | | |
| Chakola, NSW | 97.97 | 100.00 | 100.00 | 100.00 | 100.00 | No Lice |
| (327 Merino | | | | | | |
| wethers) | | | | | | |
| York, WA (305 | 99.97 | 100.00 | 100.00 | 100.00 | 100.00 | No Lice |
| Merino ewes) | | | | | | |
| Lochaber, SA | 99.97 | 99.97 | 100.00 | 100.00 | 100.00 | No Lice |
| (400 Merino | | | | | | |
| wethers) | | | | | | |
| Forbes, NSW | 99.52 | 100.00 | 100.00 | 100.00 | 100.00 | No Lice |
| (358 Merino | | | | | | on 24 of |
| ewes) | | | | | | 25 sheep* |

Note:
*4 lice infested stray sheep found in flock at the 150 day lice count which could have re-infested the one sheep on which lice were found.

### Safety

No adverse affects were reported for any sheep treated in the field trials.

### Wool Residues Study

Analysis of core samples collected from the Crookwell and Chakola field trial sites approximately 12 months post-treatment contained abamectin residues of 0.012 and 0.02 mg/kg respectively.

Analysis of core samples collected from the York, Lochaber and Forbes field trial sites approximately 12 months post-treatment contained abamectin residues of 0.054, 0.004 and 0.006 mg/kg respectively.

### Example 4

An aqueous micellar formulation containing the following ingredients was prepared:

| Ingredient | Amount (g/L) |
|---|---|
| Ivermectin | 16.00 |
| Propylene Glycol USP | 200.00 |
| Teric BL8 (a fatty alcohol alkoxylate) | 400.00 |
| Disodium Hydrogen Orthophosphate—anhydrous | 0.90 or qs |
| Sodium Dihydrogen Orthophosphate—anhydrous | 7.83 or qs |
| Pyrazole-3-carboxylic acid, | 0.023 |
| 5-hydroxy-1-(p-sulfophenyl)-4-(p- | |
| sulfophenyl)azo-, trisodium salt | |
| (e.g. Tartrazine Powder) | |
| Purified Water | 414.00 or |
| | qs to 1 L |

The formulation was prepared by mixing the surfactant (Teric BL8) and co-solvent (propylene glycol USP), dissolving the active ingredient (ivermectin) in the mixture of the surfactant and co-solvent, and then adding the water in which the buffer (disodium hydrogen orthophosphate/sodium dihydrogen orthophosphate) and colourant (Tartrazine powder) had been dissolved.

This concentrate formulation was diluted with water (in a ratio of water:concentrate formulation of 2.2:1 by volume) to 5.0 g/L ivermectin prior to use.

### Nematode Efficacy Study

The diluted formulation was used in a study of efficacy against common cattle nematodes.

The study was conducted on young cattle with a high mixed natural infestation of nematodes on a commercial grazing property at Bundarra, NSW. In the trial, 10 young Hereford steers were used. The formulation was administered as a pour-on at a single dose of 0.5 mg ivermectin per kg body weight (1 mL/10 kg).

Efficacy was measured by means of faecal egg count reductions, samples for faecal egg counts were collected on days −7, 0, 7, 14 and 21. Based on Geometric Means, the efficacy of the treatments (relative to day 0) on days 7, 14 and 21 was 98.6, 96.6 and 92.0.

The trial therefore demonstrated that the pour-on application of the formulation, at a dose of 0.5 mg ivermectin per kg body weight, provided short term control of cattle nematodes.

### Buffalo Fly Efficacy Study

A single stall trial was conducted to determine the effectiveness of the formulation against buffalo fly (Haematobia irritans exigua) on cattle. In the trial, six 8-12 month old Droughtmaster steers forming two groups of three were used.

On day 0 of the trial one group was treated with the diluted formulation containing 5.0 g/L ivermectin. Each animal received 0.5 mg ivermectin per kg body weight (1 mL/10 kg) applied as a pour-on to the top line in a narrow strip from the withers to tail head. The second group remained untreated.

On days −7, 0, 7, 14, 21, 28, 35 and 42, 1500 buffalo flies were released into each of the two stalls containing the two groups. Four days after release any remaining flies were recovered and counted to determine the efficacy of the treatment.

The percentage control in the treated group relative to the untreated group on days 4, 11, 18, 25, 32, 39 and 46 was 100, 100, 100, 100, 80.5, 93.2 and 8.1% respectively.

This trial therefore shows that the pour-on application of the formulation provided control of buffalo fly on cattle.

It will be appreciated by persons skilled in the art that numerous variations and/or modifications may be made to the specific embodiments described herein without departing from the spirit or scope of the invention as broadly described. The specific embodiments described herein are, therefore, to be considered in all respects as illustrative and not restrictive.

As used herein, the singular forms “a”, “an” and “the” include plural aspects unless the context clearly indicates otherwise. Thus, for example, reference to “a macrocyclic lactone” includes a single macrocyclic lactone as well as two or more macrocyclic lactones.

As used herein, a reference to the “control” of a parasite in or on an animal refers to treating or preventing a parasite infestation in or on the animal, and includes:
(a) preventing a parasite infestation from occurring in or on the animal;
(b) inhibiting or hindering a parasite infestation in or on the animal by retarding the spread of the parasites or retarding the growth in the number of the parasites in or on the animal; or
(c) removing or reducing the parasite infestation in or on the animal (i.e. eliminating the parasite from the animal or reducing the number of parasites in or on the animal).

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. A method for the concurrent control of internal and external parasites in and on an animal, the method comprising administering to the animal by localised topical application an aqueous micellar formulation comprising a macrocyclic lactone.

2. The method according to claim 1, wherein the formulation comprises a macrocyclic lactone, a surfactant, a water-miscible co-solvent and water, and wherein water constitutes at least 50% by weight of the total amount of water and water-miscible co-solvent in the formulation.

3. The method according to claim 1, wherein the formulation comprises:
50-300 g/L water-miscible co-solvent;
50-450 g/L surfactant;
250-890 g/L water.

4. The method according to claim 3, wherein the formulation comprises:
150-200 g/L water-miscible co-solvent;
300-420 g/L surfactant;
350-500 g/L water.

5. The method according to claim 1, wherein the macrocyclic lactone is selected from the avermectins and milbemycins.

6. The method according to claim 1, wherein the formulation comprises two or more macrocyclic lactones.

7. The method according to claim 1, wherein more than 2.0 mg of macrocyclic lactone per kg bodyweight is administered to the animal.

8. The method according to claim 7, wherein more than 4.0 mg of macrocyclic lactone per kg bodyweight is administered to the animal.

9. The method according to claim 1, wherein the internal parasite is a nematode.

10. The method according to claim 1, wherein the external parasite is a tick, lice or fly.

11. The method according to claim 1, wherein the formulation is administered to the animal in an amount of less than 3 mL per kg bodyweight.

12. The method according to claim 1, wherein the localised topical application comprises applying the formulation in 1, 2 or 3 bands on the back of the animal.

13. The method according to claim 1, wherein the animal is a sheep.

14. An aqueous micellar formulation for localised topical application to an animal for the concurrent control of internal and external parasites in and on the animal, the formulation comprising a macrocyclic lactone, a fatty alcohol alkoxylate, a water-miscible co-solvent and water, wherein water constitutes at least 50% by weight of the total amount of water and water-miscible co-solvent in the formulation.

15. The formulation according to claim 14, wherein the formulation comprises:
50-300 g/L water-miscible co-solvent;
50-450 g/L fatty alcohol alkoxylate;
250-890 g/L water.

16. The formulation according to claim 14, wherein the formulation comprises from 250 to 450 g/L fatty alcohol alkoxylate.

17. The formulation according to claim 14, wherein the formulation further comprises one or more of a buffer, preservative or colouring agent.

18. The formulation according to claim 14, wherein the formulation comprises two or more macrocyclic lactones.

19. An aqueous micellar formulation comprising:

| Ingredient | Amount (g/L) |
|---|---|
| Propylene Glycol | 187.50 ± 10% |
| Fatty alcohol alkoxylate | 375.00 ± 10% |
| Abamectin | 6.00 ± 10% |
| Sodium Dihydrogen Orthophosphate | 7.83 ± 10% |
| Preservative | 1 to 5 |
| Colouring agent | 0 to 5 |
| Water | qs to 1 L |

where the amount of the sodium dihydrogen orthophosphate is calculated based on the weight of the anhydrous salt.

20-23. (canceled)
